(19) United States
(12) Patent Application Publication
(10) Pub. No.: US 2007/0254830 A1
(43) Pub. Date: Nov. 1, 2007

(54) Interaction of Colon Cancer Related Gene C200RF20 With P120

(75) Inventors: Yusuke Nakamura, Bunkyo-ku (JP); Yoichi Furukawa, Bunkyo-ku (JP); Shuichi Nakatsuru, Kawasaki-shi (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE UNIVERSITY OF TOKYO, Bunkyo-ku (JP)

(21) Appl. No.: 11/573,338

(22) PCT Filed: Aug. 9, 2005
(86) PCT No.: PCT/JP05/14886

§ 371 Date: Feb. 6, 2007

Related U.S. Application Data
(60) Provisional application No. 60/600,602, filed on Aug. 10, 2004.

(51) Int. Cl. A61K 48/00; G01N 33/53

(52) U.S. Cl. 514/2; 435/7.1

(57) ABSTRACT

The present invention provides methods and kits for identifying inhibitors of the interaction between C20orf20 and p120 which find utility in the treatment and prevention of colon cancer. Also disclosed herein are compositions for treating or preventing colon cancer identified by the screening method of the present invention and methods of using same in the treatment and prevention of colon cancer.

### FIELD OF THE INVENTION

The present invention relates to methods and kits for identifying compounds useful in the treatment and prevention of colon cancer as well as methods and compositions for treating and preventing colon cancer. More particularly, the present method relates to the discovery that C20orf20, a colon cancer specific gene up-regulated in colorectal cancer (see PCT Publication No. WO 2004/021010, incorporated by reference herein in its entirety), interacts with p120, a proliferation-associated protein.

### BACKGROUND OF THE INVENTION

Colorectal carcinomas are leading causes of death worldwide. In spite of recent progress and therapeutic strategies, prognosis of patients with advanced cancers remains poor. Although molecular studies have revealed that alteration of tumor suppressor genes and/or oncogenes is involved in their carcinogenesis, the precise mechanisms remain to be fully elucidated.

### SUMMARY OF THE INVENTION

The present invention is based on the finding that C20orf20 and p120 interact in vivo. In view of that data and C20orf20's expression is associated with colon cancer (see, e.g., PCT Publication No. WO 2004/021010), the present invention provides methods of screening for compounds to treat colon cancer by identifying compounds that inhibit the binding of C20orf20 to p120.

Moreover, it has been discovered that C20orf20 and p120 co-precipitate in immunoprecipitation experiments and, further, that the precipitate has histone acetyltransferase activity. Accordingly, the present invention provides methods of identifying compounds for preventing or treating colon cancer by identifying compounds that modulate the histone acetyltransferase activity.

Accordingly, it is an objective of the present invention is to provide methods of screening for compounds useful in treating and preventing colon cancer.

In one embodiment, the method of the present invention comprises the steps of:
(a) contacting a polypeptide comprising a p120-binding domain of a C20orf20 polypeptide with a polypeptide comprising a C20orf20-binding domain of a p120 polypeptide in the presence of a test compound;
(b) detecting binding between the polypeptides; and
(c) selecting a test compound that inhibits binding between the polypeptides.

In some embodiments, the polypeptide comprising the p120-binding domain may comprise a C20orf20 polypeptide. Likewise, the polypeptide comprising the C20orf20-binding domain may comprise a p120 polypeptide.

In some embodiments, the polypeptide comprising the p120-binding domain is expressed in a living cell.

In some embodiments, the binding between the polypeptides is detected by a method comprising the step of detecting:
(a) an association between the polypeptide comprising the p120-binding domain and the polypeptide comprising the C20orf20 binding domain;
(b) the stabilization of p120; or
(c) the histone acetyltransferase activity of p120 associated with C20orf20.

The present invention also provides kits for screening for a compound useful in treating or preventing colon cancer. In some embodiments, the kit comprises:
(a) a first polypeptide comprising a p120-binding domain of a C20orf20 polypeptide;
(b) a second polypeptide comprising a C20orf20-binding domain of a p120 polypeptide, and
(c) a reagent that detects the interaction between the first and second polypeptides.

In some embodiments, the first polypeptide, i.e., the polypeptide comprising the p120-binding domain, comprises a C20orf20 polypeptide. Likewise, in some embodiments, the second polypeptide, i.e., the polypeptide comprising the C20orf20-binding domain, comprises a p120 polypeptide.

In some embodiments, the polypeptide comprising the p120-binding domain is expressed in a living cell.

In some embodiments, the reagent that detects the interaction between the first and second polypeptides comprises a reagent that detects:
(a) an association between the polypeptide comprising the p120-binding domain and the polypeptide comprising the C20orf20 binding domain;
(b) the stabilization of p120; or
(c) the histone acetyltransferase activity of p120 associated with C20orf20.

The present invention also provides methods of screening for a compound useful in treating or preventing colon cancer comprising the steps of:
(a) contacting a test compound to a p120 polypeptide associated with a C20orf20 polypeptide;
(b) detecting the histone acetyltransferase activity of the p120 polypeptide associated with C20orf20 polypeptide; and
(c) selecting a test compound that inhibits the histone acetyltransferase activity of the p120 polypeptide associated with C20orf20 polypeptide as compared to an activity detected in the absence of the test compound.

The present invention also provides methods for treating or preventing colon cancer in a subject comprising the step of administering a pharmaceutically effective amount of a compound selected by a method comprising the steps of:
(a) contacting a polypeptide comprising a p120-binding domain of a C20orf20 polypeptide with a polypeptide comprising a C20orf20-binding domain of a p120 polypeptide in the presence of a test compound;
(b) detecting binding between the polypeptides; and
(c) selecting a test compound that inhibits the binding between the polypeptides; wherein the binding between the polypeptides is detected by a method comprising the step of detecting:
   (i) an association between the polypeptide comprising the p120-binding domain and the polypeptide comprising the C20 or 20 binding domain;
   (ii) the stabilization of p120; or
   (iii) the histone acetyltransferase activity of p120 associated with C20orf20.

The present invention also provides methods for treating or preventing colon cancer in a subject. In some embodiments, the method comprises the step of administering a pharmaceutically effective amount of a compound that inhibits binding between a C20orf20 polypeptide and a p120 polypeptide. In some embodiments, the method comprises the step of administering a pharmaceutically effective amount of a compound that inhibits the histone acetyltransferase activity of a p120 polypeptide associated with a C20orf20 polypeptide.

The present invention also provides compositions for treating or preventing colon cancer. In some embodiments, the composition comprises a pharmaceutically acceptable carrier and a pharmaceutically effective amount of a compound selected by the method the steps of:
(a) contacting a polypeptide comprising a p120-binding domain of a C20orf20 polypeptide with a polypeptide comprising a C20orf20-binding domain of a p120 polypeptide in the presence of a test compound;
(b) detecting binding between the polypeptides; and
(c) selecting a test compound that inhibits binding between the polypeptides; wherein the binding between the polypeptides is detected by a method comprising the step of detecting:
(i) an association between the polypeptides comprising the p120-binding domain and the polypeptide comprising the C20orf20 binding domain;
(ii) the stabilization of p120; or
(iii) the histone acetyltransferase activity of p120 associated with C20orf20.

In some embodiments, the composition comprises a pharmaceutically effective amount of a compound that inhibits the binding between a C20orf20 polypeptide and a p120 polypeptide, and a pharmaceutically acceptable carrier. In other embodiments, the composition comprises a pharmaceutically effective amount of a compound that inhibits the histone acetyltransferase activity of a p120 polypeptide associated with a C20orf20 polypeptide, and a pharmaceutically acceptable carrier.

These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention.

### BRIEF DESCRIPTION OF THE DRAWINGS

FIG. 1 illustrates the interaction between C20orf20 and p120 in vivo. Immunoprecipitation with an anti-HA antibody followed by Western blot analysis with an anti-myc antibody revealed a band corresponding to C20orf20 (upper panel), and immunoprecipitation with anti-myc antibody followed by Western blot analysis with anti-HA showed a band corresponding to HA-tagged p120 (lower panel).

FIG. 2 illustrates the subcellular localization of C20orf20 and p120. Subcellular localization of exogenous HA-tagged p120 protein was analyzed by immunohistochemical staining in the absence (left panels) or presence of C20 or 20 (right panels). The localization of p120 was changed from cytoplasm to nucleus by co-transfection with C20orf20.

FIG. 3 illustrates the enhanced protein stability of the p120 protein by C20orf20. FIG. 3a illustrates increased expression of the p120 protein by C20orf20. Expression of the p120 protein in the HEK293 cells transfected with HA-tagged p120 (left panel) and cells transfected with HA-tagged p120 and MycHis-tagged C20orf20 (right panel) were analyzed by immunoblot analysis. Expression of β-actin served as an internal control. FIG. 3b illustrates the unaltered expression of p120 mRNA. Northern blot analysis of p120 was carried out using cells transfected with p120 with or without C20orf20. Expression of β-actin served as an internal control. FIG. 3c illustrates the effect of C20orf20 on the stability of the p120 protein. p120 protein stability was examined by Western blot analysis using exogenous HA-tagged p120 from in the cells transfected with or without MG132 (lane 2 and 3). Western blots were performed with anti-HA antibody. Exogenous expression of myc-tagged C20orf20 increased p120 protein in the absence of MG132 (lane 3 and 5). Western blot analysis was performed with anti-HA antibody or anti-Myc antibody. Expression of β-actin served as an internal control.

FIG. 4 illustrates the results of a histone acetyltransferase assay using an immunoprecipitant of p120. FIG. 4a illustrates the results of a Western blot analysis of immunoprecipitants from cells transfected with HA-tagged p120. FIG. 4b illustrates the histone acetyltransferase activity of an immunoprecipitant from cells that were transfected with HA-tagged p120 and immunoprecipitated with anti-HA antibody. The radioactivity of [³H]-labeled acetylated histones incubated with or without the precipitant were measured by scintillation counter (cpm).

### DETAILED DESCRIPTION OF THE PREFERRED EMBODIMENTS

I. Definitions:

The words “a”, “an”, and “the” as used herein mean “at least one” unless otherwise specifically indicated.

In the context of the present invention, a “C20orf20 polypeptide” refers to a polypeptide whose expression is linked to colon cancer. See, e.g., PCT Pub. No. WO2004/021010, incorporated by reference herein in its entirety. Exemplary C20orf20 polypeptides may be substantially identical to, e.g., Genbank accession number AB085682, SEQ ID NO:2 (e.g., encoded by SEQ ID NO:1), as well as the mouse RIKEN protein (Genbank accession number XM_{—}110403).

Herein, a “p120 polypeptide” refers to a protein comprising a bromodomain at its C-terminus and small proline-rich segments. P120 polypeptides are sometimes referred in the scientific literature as “SMAP” or “thyroid hormone interacting protein.” See, e.g., Nielsen et al., Biochim. Biophys. Acta 1306:14-16 (1996); Monden et al., J. Biol. Chem. 272:29834-29841 (1997). Exemplary p120 polypeptides may be substantially identical to, e.g., SEQ ID NO:4 (encoded by SEQ ID NO:3), and Genbank accession number AF016270.

In the context of the present invention, “inhibition of binding” between two proteins refers to at least reducing binding and sometimes completely preventing binding between the proteins. In some cases, the percentage of binding pairs in a sample will be decreased as compared to an appropriate (e.g., not treated with test compound, or from a non-cancer sample, or from a cancer sample) control. The reduction in the amount of proteins bound may be, e.g., less than 90%, 80%, 70%, 60%, 50%, 40%, 25%, 10%, 5%, 1% or less (e.g., 0%), than the pairs bound in a control sample.

The term “test compound” refers to any (e.g., chemically or recombinantly-produced) molecule that may disrupt protein-protein interaction between C20orf20 and p120, as discussed in detail herein. In some embodiments, the test compounds have a molecular weight of less than 1,500 daltons, and in some cases less than 1,000, 800, 600, 500, or 400 daltons.

A “pharmaceutically effective amount” of a compound is a quantity that is sufficient to treat and/or ameliorate a C20orf20-mediated disease in an individual. An example of a pharmaceutically effective amount may an amount needed to decrease interaction between C20orf20 and p120 when administered to an animal, so as to thereby reduce or prevent colon cancer. The decrease in interaction may be, e.g., at least about a 5%, 10%, 20%, 30%, 40%, 50%, 75%, 80%, 90%, 95%, 99%, or 100% change in binding.

The phrase “pharmaceutically acceptable carrier” refers to an inert substance used as a diluent or vehicle for a drug.

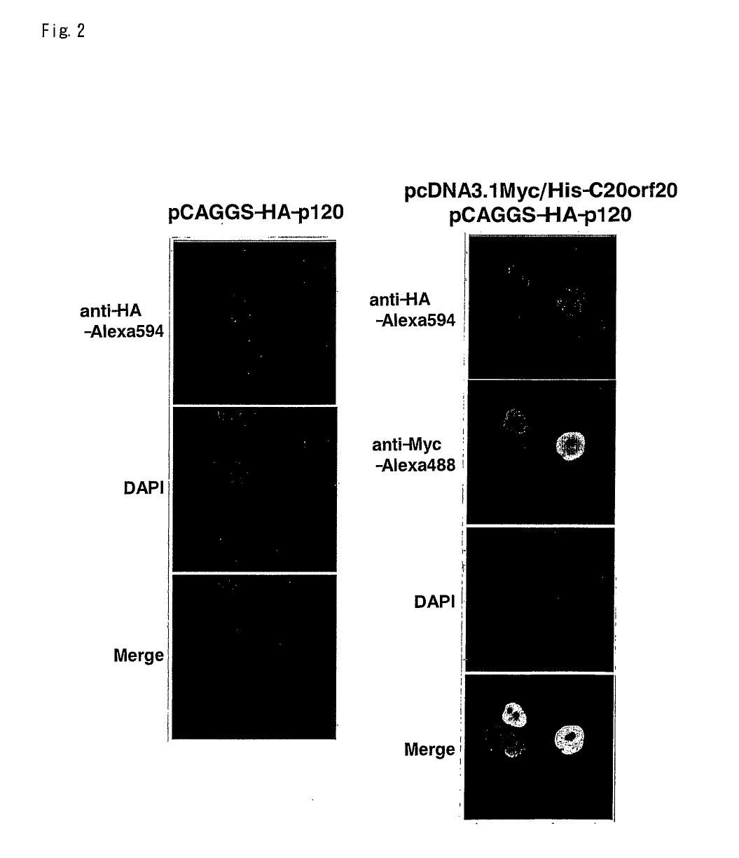

In the present invention, the term “functionally equivalent” means that the subject polypeptide has a biological activity of a reference polypeptide. For example, a functional equivalent of C20orf20 would have the histone acetyltransferase activity like wild type C20orf20. It is well known to determine the histone acetyltransferase activity of a polypeptide (see, for example, Ogryzko V V, et al., Cell. Vol. 87, 953-959, 1996, in particular FIG. 2 and Experimental procedures). For the determination, an immunoprecipitate between HA-tagged-p120 and functional equivalent of C20orf20 can be used. Furthermore, a functional equivalent of C20orf20 of the present invention may also have ability to change subcellular localization of p120. For example, it was confirmed that C20orf20 transports p120 from the cytoplasm to nucleus. Alternatively, a functional equivalent of C20orf20 of the present invention may also have ability to enhance the stability of p120 (see experimental section).

The terms “isolated” and “biologically pure” refer to material which is substantially or essentially free from components which normally accompany it as found in its native state. However, the term “isolated” is not intended to refer to the components present in an electrophoretic gel or other separation medium. An isolated component is free from such separation media and in a form ready for use in another application or already in use in the new application/milieu.

The phrase “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicitly described in each disclosed sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” wherein the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins (1984)).

In the context of the present invention, a “percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1981) Adv. Appl. Math. 2:482-489, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-7). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “small organic molecules” refers to molecules of a size comparable to those organic molecules generally used in pharmaceuticals. The term excludes biological macromolecules (e.g., proteins, nucleic acids, etc.). Preferred small organic molecules range in size up to about 5000 Da, e.g., up to 2000 Da, or up to about 1000 Da.

The terms “label” and “detectable label” are used herein to refer to any composition detectable by spectroscopic, photochemical, biochemical, immunochemical, electrical, optical or chemical means. Such labels include biotin for staining with labeled streptavidin conjugate, magnetic beads (e.g., DYNABEADS™), fluorescent dyes (e.g., fluorescein, Texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241. Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

The term “antibody” as used herein encompasses naturally occurring antibodies as well as non-naturally occurring antibodies, including, for example, single chain antibodies, chimeric, bifunctional and humanized antibodies, as well as antigenbinding fragments thereof, (e.g., Fab′, F(ab′)₂, Fab, Fv and rIgG). See also, Pierce Catalog and Handbook, 1994-1995 (Pierce Chemical Co., Rockford, Ill.). See also, e.g., Kuby, J., Immunology, 3^{rd} Ed., W.H. Freeman & Co., New York (1998). Such non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies are well known to those skilled in the art (Winter and Harris, Immunol. Today 14:243-246 (1993); Ward et al., Nature 341:544-546 (1989); Harlow and Lane, Antibodies: a laboratory manual, Cold Spring Harbor, N.Y., 1988; Hilyard et al., Protein Engineering: A practical approach (IRL Press 1992); Borrebaeck, Antibody Engineering, 2d ed. (Oxford University Press 1995); each of which is incorporated herein by reference).

The term “antibody” includes both polyclonal and monoclonal antibodies. The term also includes genetically engineered forms such as chimeric antibodies (e.g., humanized murine antibodies) and heteroconjugate antibodies (e.g., bispecific antibodies). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Holliger et al. (1993) Proc Natl Acad Sci USA. 90:6444, Gruber et al. (1994) J Immunol 152:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

Typically, an antibody has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs has been defined. The sequences of the framework regions of different light or heavy chains are relatively conserved within a species. The framework region of an antibody, that is the combined framework regions of the constituent light and heavy chains, serves to position and align the CDRs in three dimensional spaces.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. References to “V_{H}” refer to the variable region of an immunoglobulin heavy chain of an antibody, including the heavy chain of an Fv, scFv, or Fab. References to “V_{L}” refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-327 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

The terms “epitope” and “antigenic determinant” refer to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, [symbol] -carboxyglutamate, and O-phosphoserine. Amino acid analogs refers to compounds that have the same basic chemical structure as a naturally occurring amino acid, e.g., an [symbol] carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by their commonly known three letter symbols or by the one-letter symbols recommended by the IUPAC-IUB Biochemical Nomenclature Commission. Nucleotides, likewise, may be referred to by their commonly accepted single-letter codes.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operable linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

Unless otherwise defined, all technical and scientific terms used herein have the same meaning as commonly understood by one of ordinary skill in the art to which this invention belongs. In case of conflict, the present specification, including definitions, will control.

II. Producing and Identifying Compounds to Treat C20orf20-Mediated Disease

In view of the evidence provided in the examples, one aspect of the invention involves identifying test compounds that reduce or prevent the binding between C20orf20 and p120 or that reduce the histone acetyltransferase activity of p120, optionally when associated with C20orf20.

Methods for determining C20orf20/p120 binding include any methods for determining the interaction of two proteins. Such assays include, but are not limited to, traditional approaches, such as, cross-linking, co-immunoprecipitation, and co-purification through gradients or chromatographic columns. In addition, protein-protein interactions can be monitored by using a yeast-based genetic system described by Fields and co-workers (Fields and Song, Nature 340:245-246 (1989); Chien et al., Proc. Natl. Acad. Sci. USA 88, 9578-9582 (1991)) and as disclosed by Chevray and Nathans (Proc. Natl. Acad. Sci. USA 89:5789-5793 (1992)). Many transcriptional activators, such as yeast GALA, consist of two physically discrete modular domains, one acting as the DNA-binding domain, while the other one functioning as the transcription activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GALA-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

While the application refers to “C20orf20” or “p120,” it is understood that where the interaction of the two is analyzed or manipulated, it is possible to use the binding portions of one or both of the proteins in place of the full-length copies of the proteins. Fragments of C20orf20 that bind to p120 may be readily identified using standard deletion analysis and/or mutagenesis of C20orf20 to identify fragments that bind to p120. Similar analysis may be used to identify C20orf20-binding fragments of p120.

Methods of identifying test compounds that inhibit the histone acetyltransferase activity of p120, optionally when p120 is associated with C20orf20, may be performed by contacting p120 (e.g., immunoprecipitated from a cell or expressed in a cell optionally also expressing C20orf20) with a compound, and then detecting histone acetyltransferase activity. Histone acetyltransferase activity may be detected by any method known in the art. For example, histone acetyltransferase activity may be detected by incubating histones and detectably labeled acetyl CoA and subsequently detecting qualitatively or quantitatively, whether the histones are labeled.

Alternatively, the interactions of two proteins can be determined by assaying for histone acetyltransferase activity induced by the interactions. Methods for determining histone acetyltransferase activity are well known in the art (see, for example, Ogryzko V V, et al., Cell Vol. 87, 953-959, (1996) “FIG. 2 and experimental procedures”).

As disclosed herein, any test compounds, including, e.g., proteins (including antibodies), muteins, polynucleotides, nucleic acid aptamers, and peptide and nonpeptide small organic molecules, may serve as the test compounds of the present invention. Test compounds may be isolated from natural sources, prepared synthetically or recombinantly, or any combination of the same.

For example, peptides may be produced synthetically, using solid phase techniques as described in “Solid Phase Peptide Synthesis” by G. Barany and R. B. Merrifield in Peptides, Vol. 2, edited by E. Gross and J. Meienhoffer, Academic Press, New York, N.Y., pp. 100-118 (1980). Similarly, nucleic acids can also be synthesized using the solid phase techniques, as described in Beaucage, S. L., & Iyer, R. P. (1992) Tetrahedron, 48, 2223-2311; and Matthes et al., EMBO J., 3:801-805 (1984).

Where inhibitory peptides are identified, modifications of peptides of the present invention, with various amino acid mimetics or unnatural amino acids, are particularly useful in increasing the stability of the peptide in vivo. Stability can be assayed in a number of ways. For instance, peptidases and various biological media, such as human plasma and serum, have been used to test stability. See, e.g., Verhoef et al., Eur. J. Drug Metab Pharmacokinet. 11:291-302 (1986). Other useful peptide modifications known in the art include glycosylation and acetylation.

Both recombinant and chemical synthesis techniques may be used to produce test compounds of the present invention. For example, a nucleic acid of test compound may be produced by insertion into an appropriate vector, which may be expressed when transfected into a competent cell. Alternatively, nucleic acids may be amplified using PCR techniques or expression in suitable hosts (see, e.g., Sambrook et al., Molecular Cloning: A Laboratory Manual, 1989, Cold Spring Harbor Laboratory, New York, USA).

Peptides and proteins may also be expressed using recombinant techniques well known in the art, e.g., by transforming suitable host cells with recombinant DNA constructs as described in Morrison, J. Bact., 132:349-351 (1977); and Clark-Curtiss & Curtiss, Methods in Enzymology, 101:347-362 (Wu et al., eds, 1983).

Anti-C20orf20 and Anti-p120 Antibodies

In some aspects of the present invention, test compounds are anti-C20orf20 or anti-p120 antibodies. In some embodiments, the antibodies are chimeric, including but not limited to, humanized antibodies. In some cases, antibody embodiments of the present invention will bind either C20orf20 or p120 at the interface where one of these proteins associates with the other. In some embodiments, these antibodies bind C20orf20 or p120 with a Kₐ of at least about 10⁵ mol⁻¹, 10⁶ mol⁻¹ or greater, 10⁷ mol⁻¹ or greater, 10⁸ mol⁻¹ or greater, or 10⁹ mol⁻¹ or greater under physiological conditions. Such antibodies can be purchased from a commercial source, for example, Chemicon, Inc. (Temecula Calif.), or can be raised using as an immunogen, such as a substantially purified C20orf20 or p120 protein, e.g., a human protein, or a fragment thereof. Methods of preparing both monoclonal and polyclonal antibodies from provided immunogens are well-known in the art. For purification techniques and methods for identifying antibodies to specific immunogens, see e.g., PCT/US02/07144 (WO/03/077838), the contents of which are incorporated by reference herein. Methods for purifying antibodies using, for example, antibody affinity matrices to form an affinity column are also well known in the art and available commercially (AntibodyShop, Copenhagen, Denmark). Identification of antibodies capable of disrupting C20orf20/p120 association is performed using the same test assays detailed below for test compounds in general.

Converting Enzymes

Converting enzymes may act as test compounds of the present invention. In the context of the present invention, converting enzymes are molecular catalysts that perform covalent post-translational modifications to either C20orf20, p120, or both. Converting enzymes of the present invention will covalently modify one or more amino acid residues of C20orf20 and/or p120 in a manner that causes either an allosteric alteration in the structure of the modified protein, or alters the C20orf20/p120 molecular binding site chemistry or structure of the modified protein in a manner that interferes with binding between C20orf20 and p120. Interference with binding between the two molecules refers to a decrease in the Kₐ of binding by at least 25%, 30%, 40%, 50%, 60%, 70% or more relative to the Ka of binding between the proteins measured at 30° C. and an ionic strength of 0.1 in the absence of detergents. Exemplary converting enzymes of the invention include kinases, phosphatases, amidases, acetylases, glycosidase and the like.

Constructing Test Compound Libraries

Although the construction of test compound libraries is well known in the art, the present section provides additional guidance in identifying test compounds and construction libraries of such compounds for screening for effective inhibitors of C20orf20/p120 interaction and/or C20orf20/p120 histone acetyltransferase activity.

Molecular Modeling

Construction of test compound libraries is facilitated by knowledge of the molecular structure of compounds known to have the properties sought, and/or the molecular structure of the target molecules to be inhibited, i.e., C20orf20 and p120. One approach to preliminary screening of test compounds suitable for further evaluation is computer modeling of the interaction between the test compound and its target. In the present invention, modeling the interaction between C20orf20 and/or p120 provides insight into both the details of the interaction itself, and suggests possible strategies for disrupting the interaction, including potential molecular inhibitors of the interaction.

Computer modeling technology allows the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above consists of the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al. Acta Pharmaceutica Fennica 97, 159-166 (1988); Ripka, New Scientist 54-57 (Jun. 16, 1988); McKinlay and Rossmann, Annu. Rev. Pharmacol. Toxiciol. 29, 111-122 (1989); Perry and Davies, Prog Clin Biol Res. 291:189-93(1989); Lewis and Dean, Proc. R. Soc. Lond. 236, 125-140 and 141-162 (1989); and, with respect to a model receptor for nucleic acid components, Askew, et al., J. Am. Chem. Soc. 111, 1082-1090 (1989).

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al. (1988) J. Med. Chem. 31:722; Meng et al. (1992) J. Computer Chem. 13:505; Meng et al. (1993) Proteins 17:266; Shoichet et al. (1993) Science 259:1445.

Once a putative inhibitor of C20orf20/p120 interaction has been identified, combinatorial chemistry techniques can be employed to construct any number of variants based on the chemical structure of the identified putative inhibitor, as detailed below. The resulting library of putative inhibitors, or “test compounds” may be screened using the methods of the present invention to identify test compounds of the library that disrupt C20orf20/p120 association.

Combinatorial Chemical Synthesis

Combinatorial libraries of test compounds may be produced as part of a rational drug design program involving knowledge of core structures existing in known inhibitors of the C20orf20/p120 interaction. This approach allows the library to be maintained at a reasonable size, facilitating high throughput screening. Alternatively, simple, particularly short, polymeric molecular libraries may be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of combinatorial chemical libraries is well known to those of skill in the art, and may be generated by either chemical or biological synthesis. Combinatorial chemical libraries include, but are not limited to, peptide libraries (see, e.g., U.S. Pat. No. 5,010,175, Furka, Int. J. Pept. Prot. Res. 37:487-493 (1991) and Houghten et al., Nature 354:84-86 (1991)). Other chemistries for generating chemical diversity libraries can also be used. Such chemistries include, but are not limited to: peptides (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (DeWitt et al., Proc. Natl. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidylphosphonates (Campbell et al., J. Org. Chem. 59:658 (1994)), nucleic acid libraries (see Ausubel, and Sambrook, all supra), peptide nucleic acid libraries (see, e.g., U.S. Pat. No. 5,539,083), antibody libraries (see, e.g., Vaughan et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Phase Display

Another approach uses recombinant bacteriophage to produce libraries. Using the “phage method” (Scott and Smith, Science 249:386-390, 1990; Cwirla, et al, Proc. Natl. Acad. Sci., 87:6378-6382, 1990; Devlin et al., Science, 249:404-406, 1990), very large libraries can be constructed (e.g., 10⁶-10⁸ chemical entities). A second approach uses primarily chemical methods, of which the Geysen method (Geysen et al., Molecular Immunology 23:709-715, 1986; Geysen et al. J. Immunologic Method 102:259-274, 1987; and the method of Fodor et al. (Science 251:767-773, 1991) are examples. Furka et al. (14th International Congress of Biochemistry, Volume #5, Abstract FR:013, 1988; Furka, Int. J. Peptide Protein Res. 37:487-493, 1991), Houghten (U.S. Pat. No. 4,631,211, issued December 1986) and Rutter et al. (U.S. Pat. No. 5,010,175, issued Apr. 23, 1991) describe methods to produce a mixture of peptides that can be tested as agonists or antagonists.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Screening Test Compound Libraries

Screening methods of the present invention provide efficient and rapid identification of test compounds that have a high probability of interfering with C20orf20/p120 association or p120/C20orf20 histone acetyltransferase activity. Generally, any method that determines the ability of a test compound to interfere with C20orf20/p120 association or p120/C20orf20 histone acetyltransferase activity is suitable for use with the present invention. For example, competitive and non-competitive inhibition assays in an ELISA format may be utilized. Control experiments should be performed to determine maximal binding capacity of system (e.g., contacting bound C20orf20 with p120 and determining the amount of p120 that binds to C20orf20 in the examples below).

Competitive Assay Format

Competitive assays may be used for screening test compounds of the present invention. By way of example, a competitive ELISA format may include C20orf20 (or p120) bound to a solid support. The bound C20orf20 (or p120) would be incubated with p120 (or C20orf20) and a test compound. After sufficient time to allow the test compound and/or p120 (or C20orf20) to bind C20orf20 (or p120), the substrate would be washed to remove unbound material. The amount of p120 bound to C20orf20 is then determined. This may be accomplished in any of a variety of ways known in the art, for example, by using an p120 (or C20orf20) species tagged with a detectable label, or by contacting the washed substrate with a labeled anti-p120 (or C20orf20) antibody. The amount of p120 (or C20orf20) bound to C20orf20 (or p120) will be inversely proportional to the ability of the test compound to interfere with the p120/C20orf20 association. Protein, including but not limited to, antibody, labeling is described in Harlow & Lane, Antibodies, A Laboratory Manual (1988).

In a variation, C20orf20 (or p120) is labeled with an affinity tag. Labeled C20orf20 (or p120) is then incubated with a test compound and p120 (or C20orf20), then immunoprecipitated. The immunoprecipitate is then subjected to Western blotting using an anti-p120 (or C20orf20) antibody. As with the previous competitive assay format, the amount of p120 (or C20orf20) found associated with C20orf20 (or p120) is inversely proportional to the ability of the test compound to interfere with the C20orf20/p120 association.

Non-Competitive Assay Format

Non-competitive binding assays may also find utility as an initial screen for testing compound libraries constructed in a format that is not readily amenable to screening using competitive assays, such as those described herein. An example of such a library is a phage display library (See, e.g., Barret, et al. (1992) Anal. Biochem 204, 357-364).

Phage libraries find utility in being able to produce quickly working quantities of large numbers of different recombinant peptides. Phage libraries do not lend themselves to competitive assays of the invention, but can be efficiently screened in a non-competitive format to determine which recombinant peptide test compounds bind C20orf20 or p120. Test compounds identified as binding can then be produced and screened using a competitive assay format. Production and screening of phage and cell display libraries is well-known in the art and discussed in, for example, Ladner et al., WO 88/06630; Fuchs et al. (1991) Biotechnology 9:1369-1372; Goward et al. (1993) TIBS 18:136-140; Charbit et al. (1986) EMBO J. 5, 3029-3037; Cull et al. (1992) PNAS USA 89:1865-1869; Cwirla, et al. (1990) Proc. Natl. Acad. Sci. U.S.A. 87, 6378-6382.

An exemplary noncompetitive assay would follow an analogous procedure to the one described for the competitive assay, without the addition of one of the components (C20orf20 or p 120). However, as noncompetitive formats determine test compound binding to C20orf20 or p120, the ability of test compound to bind both C20orf20 and p120 needs to be determined for each candidate. Thus, by way of example, binding of the test compound to immobilized C20orf20 may be determined by washing away unbound test compound; eluting bound test compound from the support, followed by analysis of the eluate; e.g., by mass spectroscopy, protein determination (Bradford or Lowry assay, or Abs. at 280 nm determination.). Alternatively, the elution step may be eliminated and binding of test compound determined by monitoring changes in the spectroscopic properties of the organic layer at the support surface. Methods for monitoring spectroscopic properties of surfaces include, but are not limited to, absorbance, reflectance, transmittance, birefringence, refractive index, diffraction, surface plasmon resonance, ellipsometry, resonant mirror techniques, grating coupled waveguide techniques and multipolar resonance spectroscopy, all of which are known to those of skill in the art. A labeled test compound may also be used in the assay to eliminate need for an elution step. In this instance, the amount of label associated with the support after washing away unbound material is directly proportional to test compound binding.

A number of well-known robotic systems have been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett Packard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Screening Converting Enzymes

Test compounds that are converting enzymes may be assayed in a noncompetitive format, using co-factors and auxiliary substrates specific for the converting enzyme being assayed. Such co-factors and auxiliary substrates are known to one of skill in the art, given the type of converting enzyme to be investigated.

One exemplary screening procedure for converting enzymes involves first contacting C20orf20 and/or p120 with the converting enzyme in the presence of co-factors and auxiliary substrates necessary to perform covalent modification of the protein characteristic of the converting enzyme, preferably under physiologic conditions. The modified protein(s) is then tested for its ability to bind to its binding partner (i.e., binding of C20orf20 to p120). Binding of the modified protein to its binding partner is then compared to binding of unmodified control pairs to determine if the requisite change in Kₐ noted above has been achieved.

To facilitate detection of proteins in performing the assay, one or more proteins may be labeled with a detectable label as described above, using techniques well known to those of skill in the art.

Methods for Screens

The screening embodiments described above are suitable for high through-put determination of test compounds suitable for further investigation. In particular, the screening of the present invention preferably comprises one or more of the following detection steps:
(a) detecting an association between C20orf20 and p120;
(b) detecting the stabilization of p 120;
(c) detecting the histone acetyltransferase activity of p120 associated with C20orf20; or
(d) detecting the localization of p120 in the nucleus of cells, thereby indicating an interaction with C20orf20.

Alternatively, the test compound under investigation may be added to proliferating cells and proliferation of the treated cells monitored relative to proliferation of a control population not supplemented with the test compound. Cell lines suitable for screening test compounds will be obvious to one of skill in the art provided with the teachings presented herein.

For in vivo testing, the test compound may be administered to an accepted animal model.

In the present invention, the localization of p120 was changed from cytoplasm to nucleus by co-transfection with C20orf20. Accordingly, a compound that inhibit interaction between p120 and C20orf20, thereby inhibit change of localization of p120 polypeptide associated with the C20orf20 polypeptide from cytoplasm to nucleus is useful for treating or preventing colon cancer. Therefore, the present invention provides a method of screening for a compound for treating or preventing colon cancer. An embodiment of this screening method comprises the step of:
(a) introducing p120 and C20orf20 into a cell
(b) detecting the change of localization from the cytoplasm to the nucleus of p120 polypeptide associated with C20orf20 polypeptide
(c) selecting a test compound that inhibits the change of localization of step (b).

The introduction of the gene into animal cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard, B Cell 7: 1025-37 (1994); Lamb et al., Nature Genetics 5: 22-30 (1993): Rabindran et al., Science 259: 230-4 (1993)), and so on. The genes can express protein fused with tag (e.g. HA or Myc).

The subcellular localization of C20orf20 and p120 is investigated by immunohistochemical staining. Cells are transfected with tagged-C20orf20, tagged-p120, or their combination. Image of the localization can be obtained using microscope such as fluorescence microscope.

The present invention also provides a kit comprising:

i) cell expressing both of p120 and C20orf20, and

ii) detection reagent for the p120.

In the preferred embodiment, cell expressing both of p120 and C20orf20 may be obtained by transfection with expression vector of p120 and C20orf20 gene into suitable cell line. For example, HEK293, SW480 or COS7 cells may be used as the cell line. Furthermore, the detection reagent is preferably an antibody which recognizes p120. Alternatively, when the p120 or C20orf20 is expressed as fused protein with tag, an antibody recognizing the tag fused with the protein may also be used as the detection reagent. In the kit of the present invention, the antibody may be labeled with fluorescence agent (e,g, FITC, TAMRA, or GFP).

III. Formulating Medicaments from Identified Test Compounds

Accordingly, the present invention includes medicaments and methods useful in preventing or treating colon cancer (as well as other cancers characterized by cells displaying elevated levels of C20orf20 and/or binding of C20orf20 and p120). These medicaments and methods comprise at least one test compound of the present invention identified as disruptive to the C20orf20/p120 interaction in an amount effective to achieve attenuation or arrest of disease cell proliferation. More specifically, in the context of the present invention, a therapeutically effective amount means an amount effective to prevent development of, or to alleviate existing symptoms of, the subject being treated.

Individuals to be treated with methods of the present invention include any individual afflicted with cancer, including, e.g., colon cancer characterized by elevated expression of marker protein C20orf20 or exhibiting c20orf20/p120 histone acetyltransferase activity. Such an individual can be, for example, a vertebrate such as a mammal, including a human, dog, cat, horse, cow, or goat; or any other animal, particularly a commercially important animal or a domesticated animal. For purposes of the present invention, elevated expression of marker proteins refers to a mean cellular marker protein concentration for one or both marker proteins that is at least 10%, preferably 15%, 20%, 25%, 30%, 35%, 40%, 45%, 50%, 55% or more above normal mean cellular concentration of the marker protein(s).

Determining Therapeutic Dose Range

Determination of an effective dose range for the medicaments of the present invention is well within the capability of those skilled in the art, especially in light of the detailed disclosure provided herein. The therapeutically effective dose of a test compound can be estimated initially from cell culture assays and/or animal models. For example, a dose can be formulated in animal models to achieve a circulating concentration range that includes the IC₅₀ (the dose where 50% of the cells show the desired effects) as determined in cell culture. Toxicity and therapeutic efficacy of test compounds also can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (i.e., the ratio between LD₅₀ and ED₅₀). Compounds which exhibit high therapeutic indices are preferable. The data obtained from these cell culture assays and animal studies may be used in formulating a dosage range for use in humans. The dosage of such compounds may lie within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl et al., 1975, in “The Pharmacological Basis of Therapeutics”, Ch. 1 p 1. Dosage amount and interval may be adjusted individually to provide plasma levels of the active test compound sufficient to maintain the desired effects.

Pharmaceutically Acceptable Excipients

Medicaments administered to a mammal (e.g., a human) may contain a pharmaceutically-acceptable excipient, or carrier. Suitable excipients and their formulations are described in Remington's Pharmaceutical Sciences, 16th ed., 1980, Mack Publishing Co., edited by Oslo et al. For aqueous preparations, an appropriate amount of a pharmaceutically-acceptable salt is typically used in the formulation to render the formulation isotonic. Examples of the pharmaceutically-acceptable isotonic excipients include, but are not limited to, liquids such as saline, Ringer's solution, Hanks's solution and dextrose solution. Isotonic excipients are particularly important for injectable formulations.

For transmucosal administration, penetrants appropriate to the barrier to be permeated are used in the formulation. Such penetrants are generally known in the art.

Excipients may be used to maintain the correct pH of the formulation. For optimal shelf life, the pH of solutions containing test compounds is preferably from about 5 to about 8, and more preferably from about 7 to about 7.5. The formulation may also comprise a lyophilized powder or other optional excipients suitable to the present invention including sustained release preparations such as semi-permeable matrices of solid hydrophobic polymers, which matrices are in the form of shaped articles, e.g., films, liposomes or microparticles. It will be apparent to those persons skilled in the art that certain excipients may be more preferable depending upon, for instance, the route of administration, the concentration of test compound being administered, or whether the treatment uses a medicament that includes a protein, a nucleic acid encoding the test compound, or a cell capable of secreting a test compound as the active ingredient.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen.

For oral administration, carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by formulating a test compound with a solid dispersable excipient, optionally grinding a resulting mixture and processing the mixture of granules after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Many of the compounds of the invention may be optionally provided as salts with pharmaceutically compatible counter-ions. Pharmaceutically compatible salts may be formed with many acids, including but not limited to hydrochloric, sulfuric, acetic, lactic, tartaric, malic, succinic, etc., depending upon the application. Salts tend to be more soluble in aqueous or other protonic solvents that are the corresponding free base forms.

In addition to acceptable excipients, formulations of the present invention may include therapeutic agents other than identified test compounds. For example formulations may include anti-inflammatory agents, pain killers, chemotherapeutics, mucolytics (e.g. n-acetyl-cysteine) and the like. In addition to including other therapeutic agents in the medicament itself, the medicaments of the present invention may also be administered sequentially or concurrently with the one or more other pharmacologic agents. The amounts of medicament and pharmacologic agent depend, for example, on what type of pharmacologic agent(s) is are used, the disease being treated, and the scheduling and routes of administration.

Following administration of a medicament of the invention, the mammal's physiological condition can be monitored in various ways well known to the skilled practitioner.

Gene Therapy

Protein and peptide test compounds identified as disruptors of C20orf20/p120 association may be therapeutically delivered using gene therapy to patients suffering from colon cancer. Exemplary test compounds amenable to gene therapy techniques include converting enzymes as well as peptides that directly alter the C20orf20/p 120 association by steric or allosteric interference. In some aspects, gene therapy embodiments include a nucleic acid sequence encoding a suitable identified test compound of the invention. In preferred embodiments, the nucleic acid sequence includes regulatory elements necessary for expression of the test compound in a target cell. The nucleic acid may be equipped to stably insert into the genome of the target cell (see e.g., Thomas, K. R. and Capecchi, M. R. (1987) Cell 51:503 for a description of homologous recombination cassettes vectors).

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

For general reviews of the methods of gene therapy, see Goldspiel et al., (1993) Clinical Pharmacy 12:488-505; Wu and Wu, (1991) Biotherapy 3:87-95; Tolstoshev, (1993) Ann. Rev. Pharmacol. Toxicol. 33:573-596; Mulligan, (1993) Science 260:926-932; and Morgan and Anderson, (1993) Ann. Rev. Biochem. 62:191-217; (1993) TIBTECH 11(5):155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

IV. Screening and Treatment Kits

In one embodiment, the present invention provides an article of manufacture or kit for screening for a compound useful in treating or preventing colon cancer, wherein the kit comprises: (a) a p120-binding domain of a C20orf20 polypeptide; (b) a C20orf20-binding domain of a p120 polypeptide, and (c) a reagent that detects the interaction between the two polypeptides. As discussed above, the polypeptide comprising the p120-binding domain may comprise a full length C20orf20 polypeptide or a p120-binding portion thereof. Likewise, the polypeptide comprising the C20orf20-binding domain may comprise a full-length p120 polypeptide or a C20orf20-binding portion thereof.

The reagent that detects the interaction between the two polypeptides preferably detects:
(a) an association between the polypeptide comprising the p120-binding domain and the polypeptide comprising the C20orf20 binding domain;
(b) the stabilization of p120; or
(c) the histone acetyltransferase activity of p120 associated with C20orf20.

In a further embodiment of the invention, articles of manufacture and kits containing materials useful for treating the pathological conditions described herein are provided. The article of manufacture may comprise a container of a medicament as described herein with a label. Suitable containers include, for example, bottles, vials, and test tubes. The containers may be formed from a variety of materials, such as glass or plastic. In the context of the present invention, the container holds a composition having an active agent which is effective for treating a cell proliferative disease, for example, colon cancer. In one embodiment, the active agent in the composition is an identified test compound (e.g., antibody, small molecule, etc.) capable of disrupting C20orf20/p120 association in vivo. The label on the container should indicate that the composition is used for treating one or more conditions characterized by abnormal cell proliferation. The label may also indicate directions for administration and monitoring techniques, such as those described herein.

In addition to the container described above, a kit of the invention may optionally comprise a second container housing a pharmaceutically-acceptable diluent. It may further include other materials desirable from a commercial and user standpoint, including other buffers, diluents, filters, needles, syringes, and package inserts with instructions for use.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

Hereinafter, the present invention is described in more detail by reference to the Examples. However, the following materials, methods and examples only illustrate aspects of the invention and in no way are intended to limit the scope of the present invention. As such, methods and materials similar or equivalent to those described herein can be used in the practice or testing of the present invention,

### EXAMPLES

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of non-critical parameters that could be changed or modified to yield essentially similar results.

### Example 1

### Materials and Methods

(a) Immunoprecipitation Assay with Thyroid Hormone Receptor Co-Activating Protein (p120).

The entire coding region of p120 was amplified by RT-PCR and cloned into pCAGGS-HA vector. The primer sequences used for the amplification were 5′-ATAGAATTCTCTTCTGTCATGAGAAGTGG-3′ (SEQ ID NO.5) and 5′-ATACTCGAGTCACTTTTTCATCTTC-3′ (SEQ ID NO.6). We transfected COS7 cells with pcDNA3.1Myc/His-C20orf20, pCAGGS-HA-p120 or their combination, and performed immunoprecipitation assay using anti-Myc or anti-HA antibody. Cells were washed with PBS and lysed in TNE buffer containing 150 mM NaCl, 0.5% NP40, 10 mM Tris-HCl (pH7.8), supplemented with a Protease Inhibitor Cocktail 3 (Roche). In a typical immunoprecipitation reaction, 400 μg of whole-cell extract was incubated with 1 μg of anti-Myc (Santa Cruz) or anti-HA antibody (Roche), and 20 μl of protein G Sepharose beads (Zymed) at 4° C. for 2 hr. Beads were washed four times in 1 ml of TNE buffer and proteins bound to the beads were eluted by boiling in Laemmli sample buffer. The precipitated protein was separated by SDS-PAGE and immunoblot analysis was carried out using with either rat anti-HA antibody or mouse anti-Myc antibody, respectively.

(b) Immunofluorescence and Confocal Microscopy

Cells transfected with pcDNA3.1 Mcy/His-C20orf20, pCAGGSHA-p 120, or their combination were fixed with PBS containing 4% paraformaldehyde for 15 min, then rendered permeable with PBS containing 0.1% Triton X-100 for 2.5 min at RT. Subsequently the cells were covered with 3% BSA in PBS for 20 min at room temperature to block non-specific hybridization. Mouse anti-myc monoclonal antibody (Santa Cruz) at 1:1000 dilution or rabbit anti-HA antibody (Roche) at 1:1000 dilution was used for the first antibody, and the reaction was visualized after incubation with Alexa Fluor-488 anti-mouse and Alexa Fluor-594 anti-rabbit second antibody (Molecular Probes, Eugene, Oreg.). Nuclei were counter-stained with 4′,6-diamidino-2-phenylindole, dihydrochloride (DAPI). Fluorescent images were obtained under a confocal microscope (Leica).

(c) Western Blotting

HEK293 cells transfected with pcDNA3.1Myc/His-C20orf20 and pCAGGS-HA-p120 were incubated with or without MG132 (for eight hours before the harvest), and harvested at 0, 12, 24, 36, 48, and 72 h after transfection and lysed Nonidet P40 lysis buffer (10 mM Tris-HCl pH 7.5, 150 mM NaCl, 0.5% NP40), supplemented with a Protease Inhibitor Cocktail 3 (CALBIOCHEM). After the cells were homogenized and centrifuged at 15,000 rpm for 20 min, the supernatant were standardized for protein concentration by Lowry assay (Bio-Rad). Proteins were separated by 8% SDS-PAGE and immunoblotted with mouse anti-myc (Santa Cruz), rat anti-HA (Roche), or mouse anti beta-actin antibody (Sigma). HRP-conjugated goat anti-mouse IgG (Amersham) and goat anti-rat IgG served as the secondary antibody for the ECL Detection System (Amersham).

(d) Northern Blotting

Total RNAs were extracted from HEK293 cells at 0, 12, 24, 36, and 48 hr after transfection of pcDNA3.1myc/His-C20orf20 and pCAGGS-HA-p120. These RNAs were isolated using TRIZOL reagent (GIBCO-BRL). A 5 ug aliquot of each total RNA was separated on a 1% agarose gel containing 1× morpholinepropanesulfonic acid buffer and 2% formaldehyde, and transferred to a nylon membrane. The blots were hybridized with a random-primed ³²P-labeled p120 and C20orf20.

(e) Histone Acetyltransferase Assay

An HA-tagged p120 protein was prepared by immunoprecipitation with anti-HA antibody-conjugated to agarose. Approximately 5×10⁷ HEK293F cells transfected with pcDNA3.1Myc/His-C20orf20 and pCAGGS-HA-p120 were washed with PBS and lysed in 2 ml of TNE buffer containing 150 mM NaCl, 0.5% NP-40, 10 mM Tris-HCl (pH7.8), supplemented with a Protease Inhibitor Cocktail 3 (Roche). In a typical immunoprecipitation reaction, the whole-cell extract was incubated with 100 μl of anti-HA agarose conjugate (SIGMA) at 4° C. for 2 hr. Beads were washed five times in 1 ml of TNE buffer and proteins bound to the beads were eluted by HA peptide (SIGMA). The precipitated protein was quantified by Lowry method (Bio Rad). 10 μg of precipitated protein was incubated with 33 μg/ml thymus histones (Sigma Chemical Co.) and 6 μmol of [³H]-labeled acetyl CoA (4.3mCi/mmol, Amersham Life Science Inc.), in 30 μl of buffer containing 50 mM Tris-HCl (pH 8.0), 10% glycerol, 1 mM DTT, 10 mM sodium butylate, at 30° C. for 30 min. Labeled histones were transferred onto Whatman P-81 phosphocellulose filter paper and washed twice with 0.2 M sodium carbonate buffer (pH 9.2) at room temperature for 5 min. The radioactivity was counted in a liquid scintillation counter.

### Example 2

### The Association Between C20orf20 and p120 In Vivo

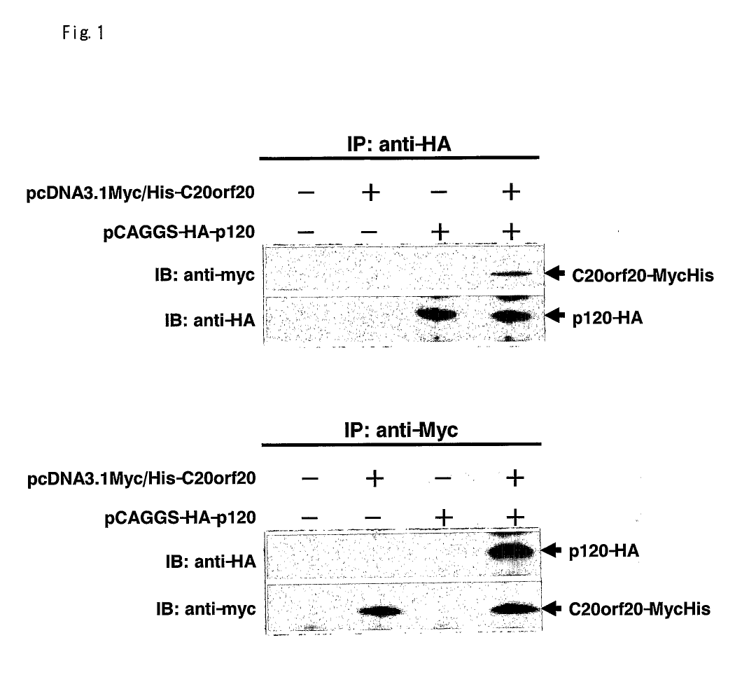

To prove the association between C20orf20 and p120 in vivo, an immunoprecipitation assay was carried out using COS7 cells transfected with plasmids expressing pcDNA3.1Myc/His-C20orf20, pCAGGS-HA-p120, or their combination. Immunoprecipitation with anti-HA antibody and subsequent Western blot analysis with anti-myc antibody revealed a single band corresponding to Myc/His-tagged C20orf20 (FIG. 1, upper panel). On the other hand, immunoprecipitation with anti-myc antibody and subsequent Western blot analysis with anti-HA showed a band corresponding to HA-tagged p120 (FIG. 1, lower panel). These data suggest that C20orf20 associates with p120 in vivo.

### Example 3

### Altered Subcellular Localization of p120 in the Presence of C20orf20

The subcellular localization of C20orf20 and p120 was further investigated by immunohistochemical staining. SW480 cells were transfected with pcDNA3.1Myc/His-C20orf20, pCAGGS-HA-p120, or their combination. As shown previously, exogenous Myc-tagged C20orf20 protein was stained in the nucleus. On the other hand, exogenous HA-tagged p120 protein was slightly stained in the cytoplasm of the cells transfected with pCAGGS-HA-p120 alone (FIG. 2, left panel). Interestingly, HA-tagged p120 protein co-localized with C20orf20 in the nuclei of the cells transfected with pCAGGS-HA-p120 and pcDNA3.1Myc/His-C20orf20 (FIG. 2, right panel). In addition, the staining of HA-tagged p120 in the nuclei was significantly higher than that in the cytoplasms, suggesting increased protein stability by the change of subcellular localization.

### Example 4

### Increased Protein Stability of p120 by C20orf20

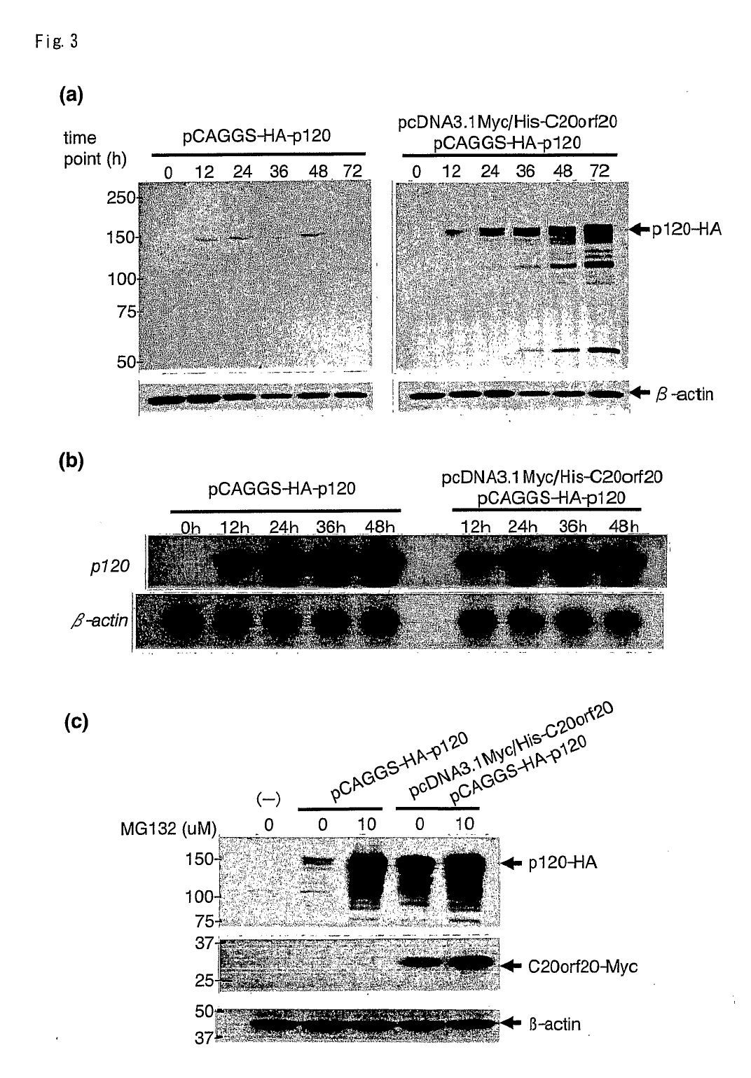

To examine the protein stability, pCAGGS-HA-p120 was transfected with or without pcDNA3.1Myc/His-C20orf20 into HEK293 cells, and Western and Northern blot analyses were carried out. The Western blot analysis using extracts from cells transfected with pCAGGS-HA-p120 and those co-transfected with pCAGGS-HA-p120 and pcDNA3.1Myc/His-C20orf20 revealed that expression of exogenous p120 protein was markedly increased in the presence of C20orf20 (FIG. 3a). Northern blot analysis using the cells disclosed that expression of p120 messenger RNA was unchanged by the co-expression of C20orf20 (FIG. 3b). To examine the possibility of post-transcriptional modulation, the effect of MG132, a protease inhibitor, was tested on the protein stability of p120. As shown FIG. 3c, MG132 markedly accumulated p120 in the absence of C20orf20 (lane 2 and 3), indicating that p120 is degraded quickly in the absence of MG132. Co-transfection of pcDNA3.1Myc/His-C20orf20 with pCAGGS-HA-p120, enhanced p120 stabilization in the absence of MG132 (FIG. 3c, lane 4 and 5), implying that C20orf20 is involved in stabilization or degradation of p120.

### Example 5

### Histone Acetyltransferase Activity of p120

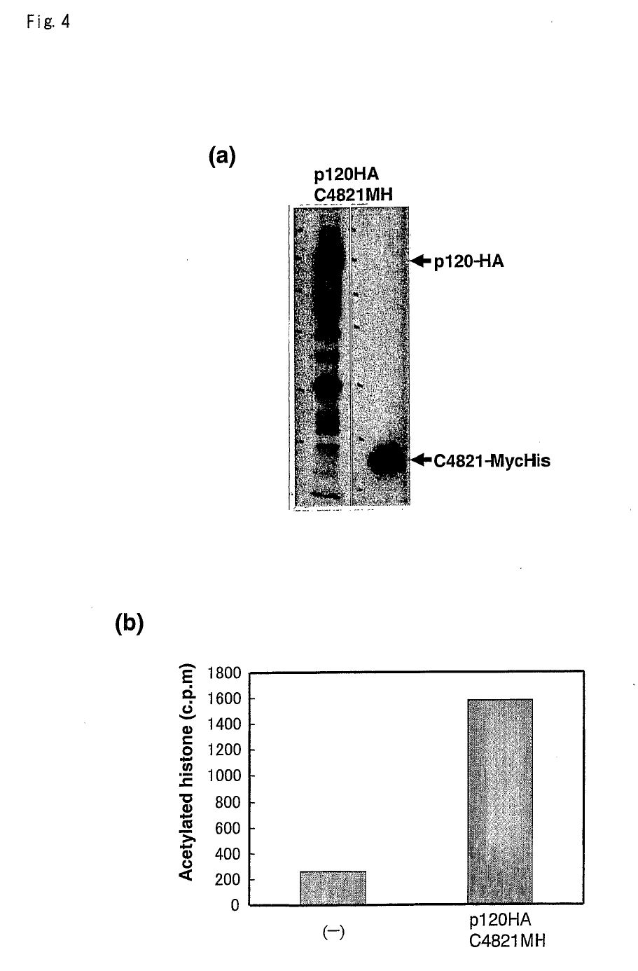

Since bromodomain containing proteins such as p300/CBP, TAF250 and Gcn5p have histone acetylase activity and function as transcriptional activators, the activity of p120 was explored. HA-tagged p120 protein was immunoprecipitated from cells transfected with both pcDNA3.1Myc/His-C20orf20 and pCAGGS-HA-p120. Immunoblot analysis demonstrated that HA-tagged p120 and myc-tagged C20orf20 were included in the precipitant (FIG. 4a). A mixture of histones was incubated with the precipitants together with ³H-labeled acetyl CoA. As a result, the radioactivity of histones incubated with the precipitants was significantly higher than that incubated with precipitants from mock cells (FIG. 4b). This data indicates that p120 or protein(s) associated with p120 have histone acetyl transferase activity. Therefore, C20orf20 that associates with p120 affects the histone acetyltransferase activity and modulate transcriptional activity mediated by p120.

The above examples are provided to illustrate the invention but are not intended to limit its scope. Other variants of the invention will be readily apparent to one of ordinary skill in the art and are encompassed by the appended claims.

### INDUSTRIAL APPLICABILITY

The present inventors have shown that C20orf20 interacts with p120, and the inhibition of the interaction led to inhibit of cell proliferation of colorectal cancer cells. Thus, agents that inhibit the binding between C20orf20 and p120 and prevent its activity may find therapeutic utility as anti-cancer agents, particularly anti-cancer agents for the treatment and prevention of colon cancer.

All publications, databases, Genbank sequences, patents, and patent applications cited herein are hereby incorporated by reference.

While the invention has been described in detail and with reference to specific embodiments thereof, it will be apparent to one skilled in the art that various changes and modifications can be made therein without departing from the spirit and scope of the invention, the metes and bounds of which are set by the appended claims.

1. A method of screening for a compound useful in treating or preventing colon cancer, said method comprising the steps of:
(a) contacting a polypeptide comprising a p120-binding domain of a C20orf20 polypeptide with a polypeptide comprising a C20orf20-binding domain of a p120 polypeptide in the presence of a test compound;
(b) detecting binding between the polypeptides; and
(c) selecting a test compound that inhibits binding between the polypeptides.

2. The method of claim 1, wherein the polypeptide comprising the p120-binding domain comprises a C20orf20 polypeptide.

3. The method of claim 1, wherein the polypeptide comprising the C20orf20-binding domain comprises a p120 polypeptide.

4. The method of claim 1, wherein the polypeptide comprising the p120-binding domain is expressed in a living cell.

5. The method of claim 1, wherein the binding between the polypeptides is detected by a method comprising the step of detecting:
(a) an association between the polypeptide comprising the p120-binding domain and the polypeptide comprising the C20orf20 binding domain;
(b) the stabilization of p120; or
(c) the histone acetyltransferase activity of p120 associated with C20orf20.

6. A kit for screening for a compound for treating or preventing colon cancer, wherein the kit comprises:
(a) a polypeptide comprising a p120-binding domain of a C20orf20 polypeptide;
(b) a polypeptide comprising a C20orf20-binding domain of a p120 polypeptide, and
(c) a reagent to detect the interaction between the polypeptides.

7. The kit of claim 6, wherein the polypeptide comprising the p120-binding domain comprises a C20orf20 polypeptide.

8. The kit of claim 6, wherein the polypeptide comprising the C20orf20-binding domain comprises a p120 polypeptide.

9. The kit of claim 6, wherein the polypeptide comprising the p120-binding domain is expressed in a living cell.

10. The kit of claim 6, wherein the reagent to detect the interaction between the polypeptides comprises a reagent that detects:
(a) an association between the polypeptide comprising the p120-binding domain and the polypeptide comprising the C20orf20 binding domain;
(b) the stabilization of p120; or
(c) the histone acetyltransferase activity of p120 associated with C20orf20.

11. A method of screening for a compound for treating or preventing colon cancer, said method comprising the steps of:
(a) contacting a test compound to a p120 polypeptide associated with a C20orf20 polypeptide;
(b) detecting the histone acetyltransferase activity of the p120 polypeptide associated with the C20orf20 polypeptide; and
(c) selecting a test compound that inhibits the histone acetyltransferase activity of the p120 polypeptide associated with the C20orf20 polypeptide as compared to an activity detected in the absence of the test compound.

12. A method of screening for a compound for treating or preventing colon cancer, said method comprising the steps of:
(a) introducing p120 and C20orf20 into a cell
(b) detecting the change of localization from the cytoplasm to the nucleus of p120 polypeptide associated with C20orf20 polypeptide
(c) selecting a test compound that inhibits the change of localization of step (b).

13. A method for treating or preventing colon cancer in a subject, said method comprising the step of administering a pharmaceutically effective amount of the compound selected by the method of claim 1, 11 or 12.

14. A method for treating or preventing colon cancer in a subject, wherein the method comprises the step of administering a pharmaceutically effective amount of a compound that inhibits the binding between a C20orf20 polypeptide and a p120 polypeptide.

15. A method for treating or preventing colon cancer in a subject, wherein the method comprises the step of administering a pharmaceutically effective amount of a compound that inhibits the histone acetyltransferase activity of a p120 polypeptide associated with a C20orf20 polypeptide.

16. A composition for treating or preventing colon cancer, wherein the composition comprises a pharmaceutically effective amount of the compound selected by the method of claim 1, 11 or 12, and a pharmaceutically acceptable carrier.

17. A composition for treating or preventing colon cancer, wherein the composition comprises a pharmaceutically effective amount of a compound that inhibits the binding between a C20orf20 polypeptide and a p120 polypeptide, and a pharmaceutically acceptable carrier.

18. A composition for treating or preventing colon cancer, wherein the composition comprises a pharmaceutically effective amount of a compound that inhibits the histone acetyltransferase activity of a p120 polypeptide associated with a C20orf20 polypeptide, and a pharmaceutically acceptable carrier.


SEQUENCE LISTING (6 sequences)

SEQ ID NO: 1 - DNA, 1634 bp, Homo sapiens
  CDS (72)..(683)
agtgcgcctgcgcggagctcgtggccgcgcctgctcccgccgggggctccttgctcggccgggccgcggccatgggagaggccgaggtgggcggcgggggcgccgcaggcgacaagggcccgggggaggcggccaccagcccggcggaggagacagtggtgtggagccccgaggtggaggtgtgcctcttccacgccatgctgggccacaagcccgtcggtgtgaaccgacacttccacatgatttgtattcgggacaagttcagccagaacatcgggcggcaggtcccatccaaggtcatctgggaccatctgagcaccatgtacgacatgcaggcgctgcatgagtctgagattcttccattcccgaatccagagaggaacttcgtccttccagaagagatcattcaggaggtccgagaaggaaaagtgatgatagaagaggagatgaaagaggagatgaaggaagacgtggacccccacaatggggctgacgatgttttttcatcttcagggagtttggggaaagcatcagaaaaatccagcaaagacaaagagaagaactcctcagacttggggtgcaaagaaggcgcagacaagcggaagcgcagccgggtcaccgacaaagtcctgaccgcaaacagcaacccttccagtcccagtgctgccaagcggcgccgcacgtagaccctcagccctggtggcggcagagaagcgggcgaggcactgtggtcgctgagggggttggctgggtctgagtgccacccccaggccacagtgataccatcccagtgccatgagcccacactgcccgccctcaggctctcaggtgaacgtggccgtcagcggggaaacgtgtgtgtcagttggaccatgtgggaccctgatggacctgaaagaccaggatcggtccagctcagatattgagggctctgaagcctagttctgtcttctctggagcagctgtggcttccccgtggctgcttggtgacatggattagcgctacgtgggctgcagcatttgggatccaggctacctagaggggcatcgggccagggaaaacctcggattagcaagcaataaaaacatgacctcactcttcctcaaaggagcccctggtcttccctgtgtgactcagttctttccatctgtttgtcccgctgcaagcctctttctgcgctgactgtgacattggaacgtggccttcctgtcaccccctccgtgccacgcactgaaggccacccccacccacctgggaaactaagaactggatattttgcctcattcacttgtactgtaacaatgtatataatttggttggtatttcactatttaatttttaagaagcctattttactagtgttttatatgaacaaagtactgcagaagttaaacctgtgttgtattttttctgagatgttttgctttaagagatactttttgctcagtttttatatgccagatacagagaatttgtagcggttatttttgtatgatctagtaacttgcaaacagaccaaatggatgagaggcggggaccgtgcagctgtcggctgatgaggaggcggccgccccagtgctgatggagatgccactttcgtgtgactgcgaacattaaagcacaaaaaaaatcc

SEQ ID NO: 2 - protein, 204 aa, Homo sapiens
MGEAEVGGGGAAGDKGPGEAATSPAEETVVWSPEVEVCLFHAMLGHKPVGVNRHFHMICIRDKFSQNIGRQVPSKVIWDHLSTMYDMQALHESEILPFPNPERNFVLPEEIIQEVREGKVMIEEEMKEEMKEDVDPHNGADDVFSSSGSLGKASEKSSKDKEKNSSDLGCKEGADKRKRSRVTDKVLTANSNPSSPSAAKRRRT

SEQ ID NO: 3 - DNA, 3154 bp, Homo sapiens
  CDS (81)..(2843)
cgggcaaacacaagctgctaagcactggccccacagagccatggtccatccgagagaagctatgtttagcatcttctgtcatgagaagtggcgatcaaaattgggtatcagttagcagagcaatcaagccctttgcagaacctggccgccctccagactggttctctcaaaaacattgtgcttcccagtactcggagcttttagagaccactgagacaccaaaacggaaacgaggtgaaaagggagaagtggtggaaactgttgaagatgttattgttcggaaattgactgctgagcgagttgaagaactaaagaaagtgataaaggaaacccaggagagatatagacggctaaagagagatgcagaactaattcaagctggacacatggacagcagactggatgagctttgcaatgacattgcaacgaaaaagaaattggaagaagaggaggctgaagtaaagaggaaggctacagatgctgcataccaggctcgtcaagcagtaaaaacacccccccggaggttacccactgtgatggttcgctctcctatagattctgcctccccaggaggtgattatccacttggggacttgactccaaccactatggaagaggctacctctggggtaacccccgggactttgccgagtaccccagtcacctcgtttcctgggattcctgacacccttcctccaggctctgcacccttagaagcccccatgaccccagtaacagatgattcaccccagaaaaagatgcttggacagaaagcaactccacccccctcccctctgctgtcagagctcttgaagaagggcagcctcctgcctactagccccagactggtcaatgagagtgaaatggctgtggcttctggccacctgaacagtacaggtgtcctcctggaggtaggcggggtccttcccatgatacatggtggggagatacagcaaacacccaatactgttgcagcctcccctgctgcatcaggtgctcccactctttcccggcttttagaagctggtcctacacagttcaccacacctcttgcttccttcactactgttgccagtgagcctccagttaaacttgtgccaccccctgtagagtctgtgtcccacgctaccattgtcatgatgcctgcgctgccagcaccatcctctgctccggctgtctccactactgaaagtgtagctccagtgagtcaacccgacaactgtgttcccatggaggctgtgggggatccacatactgtgactgtttccatggacagcagtgaaatatccatgatcatcaattctatcaaagaagagtgttttcgatcaggggtagcagaggctcctgttggatcaaaggctcccagcatagatgggaaggaagaattagatctggctgagaagatggatattgctgtgtcttacacaggtgaagagctggattttgagactgttggagacatcattgccatcattgaggacaaggtagatgatcatcctgaagtgctggatgtggcagcagtggaagcagcactgtcattttgtgaagaaaatgatgatcctcagtccctgcctggcccctgggagcatcctatccagcaggagcgggacaagccagtacctctccctgcaccagaaatgacggtcaagcaagagagactggactttgaggaaacggaaaacaagggaatacatgaactggtggacatcagggagcccagtgcagagatcaaggtggaacctgcagaaccagagccagtcatttcaggagccgaaatagtagctggagttgttccagccacaagtatggagccaccagaactcaggagtcaggacttagatgaggaactgggaagtactgcagctggagagattgttgaagcagatgttgccattgggaaaggcgatgagactccacttacaaatgtgaagacagaggcatcccctgaaagcatgttgtctccatcacatggctcaaatcccattgaagatcctttagaggcagagactcagcacaagtttgaaatgtcagactcattgaaagaagaatcagggactatttttggaagccagataaaggatgccccaggtgaggatgaggaggaagatggtgtcagtgaagcggccagcctagaggagcctaaggaagaggatcaaggagaaggctacttgtcagaaatggataatgaacctcctgtgagcgagagtgatgatggcttcagcatacacaatgctacactgcagtcacacacactggcagactccatccccagcagccctgcttcttcacagttctctgtctgtagtgaggatcaggaagctattcaggcacagaaaatttggaagaaagccatcatgcttgtatggagagctgcagctaatcataggtatgccaatgtcttcctgcagcctgttacagatgacatagcacctggctaccacagcattgtgcagaggcctatggatttgtcaactattaagaaaaacatagaaaatggactgatccgaagcacagctgaatttcagcgtgacattatgctgatgtttcagaatgctgtaatgtacaatagctcagaccatgatgtctatcacatggcagtggagatgcagcgagatgtcttggaacagatccagcaattcttggccacgcagttgattatgcaaacatccgagtctgggatcagtgctaaaagtcttcgagggagagattctacccgcaaacaggatgcttcagagaaggacagtgtcccaatgggctctcctgccttccttctctctctctttgatggaggaaccaggggacgccgctgtgccattgaagcagatatgaagatgaaaaagtgaagcctcagagttaccctctttgagccgaacctaaaataaaagtaaacaagatagagcttgggcttgcgggcccagttccagaggtggaagttacagaagaggaggtacctgggccacacgacatgagctggaaaatctctcttagagagttggagtagcacaattgcctgttttagggcagaaaccatgggctatgttaatgtcctaatgtgtagctagcagatcgtagctagtttgtattgtcttgtcaattgtacagactttttaaaaaaaacaaccaccagtgaaatgtgtgtgtatacaataaactg

SEQ ID NO: 4 - protein, 920 aa, Homo sapiens
MRSGDQNWVSVSRAIKPFAEPGRPPDWFSQKHCASQYSELLETTETPKRKRGEKGEVVETVEDVIVRKLTAERVEELKKVIKETQERYRRLKRDAELIQAGHMDSRLDELCNDIATKKKLEEEEAEVKRKATDAAYQARQAVKTPPRRLPTVMVRSPIDSASPGGDYPLGDLTPTTMEEATSGVTPGTLPSTPVTSFPGIPDTLPPGSAPLEAPMTPVTDDSPQKKMLGQKATPPPSPLLSELLKKGSLLPTSPRLVNESEMAVASGHLNSTGVLLEVGGVLPMIHGGEIQQTPNTVAASPAASGAPTLSRLLEAGPTQFTTPLASFTTVASEPPVKLVPPPVESVSHATIVMMPALPAPSSAPAVSTTESVAPVSQPDNCVPMEAVGDPHTVTVSMDSSEISMIINSIKEECFRSGVAEAPVGSKAPSIDGKEELDLAEKMDIAVSYTGEELDFETVGDIIAIIEDKVDDHPEVLDVAAVEAALSFCEENDDPQSLPGPWEHPIQQERDKPVPLPAPEMTVKQERLDFEETENKGIHELVDIREPSAEIKVEPAEPEPVISGAEIVAGVVPATSMEPPELRSQDLDEELGSTAAGEIVEADVAIGKGDETPLTNVKTEASPESMLSPSHGSNPIEDPLEAETQHKFEMSDSLKEESGTIFGSQIKDAPGEDEEEDGVSEAASLEEPKEEDQGEGYLSEMDNEPPVSESDDGFSIHNATLQSHTLADSIPSSPASSQFSVCSEDQEAIQAQKIWKKAIMLVWRAAANHRYANVFLQPVTDDIAPGYHSIVQRPMDLSTIKKNIENGLIRSTAEFQRDIMLMFQNAVMYNSSDHDVYHMAVEMQRDVLEQIQQFLATQLIMQTSESGISAKSLRGRDSTRKQDASEKDSVPMGSPAFLLSLFDGGTRGRRCAIEADMKMKK

SEQ ID NO: 5 - DNA, 29 bp, Artificial
  Artificially synthesized primer for PCR
atagaattctcttctgtcatgagaagtgg

SEQ ID NO: 6 - DNA, 25 bp, Artificial
  Artificially synthesized primer for PCR
atactcgagtcactttttcatcttc